(19) United States
(12) Patent Application Publication
(10) Pub. No.: US 2020/0238030 A1
(43) Pub. Date: Jul. 30, 2020

(54) BRONCHOSCOPE

(71) Applicant: Lymol Medical, Inc., Woburn, MA (US)

(72) Inventor: Libor Krupica, Nashua, NH (US)

(21) Appl. No.: 16/849,377

(22) Filed: Apr. 15, 2020

Related U.S. Application Data
(62) Division of application No. 15/944,386, filed on Apr. 3, 2018.
(60) Provisional application No. 62/481,377, filed on Apr. 4, 2017.

(51) Int. Cl. A61M 16/00; A61B 1/00; A61M 16/18; A61F 2/95; A61B 18/22; A61B 1/06; A61B 1/018; A61B 1/015; A61B 1/012; A61B 1/267; A61M 16/08

(52) U.S. Cl. CPC A61M 16/0096; A61M 16/01; A61B 1/00066; A61M 16/18; A61F 2/95; A61B 18/22; A61B 1/06; A61B 1/018; A61B 1/015; A61B 1/0125; A61B 1/00128; A61B 1/0011; A61B 1/2676; A61M 16/0833; A61B 1/00105

(57) ABSTRACT

A bronchoscope for insertion into a patient's airways for diagnostic and therapeutic purposes. The bronchoscope includes a bronchoscope head configured to be releasably secured and sealed to one tube of a set of interchangeable bronchial and/or tracheal tubes.

### RELATED APPLICATION

This application is a divisional of U.S. patent application Ser. No. 15/944,386 filed on Apr. 3, 2018 entitled BRONCHOSCOPE, which claims priority from U.S. Provisional Patent Application No. 62/481,377 filed on Apr. 4, 2017 entitled BRONCHOSCOPE, which is hereby incorporated by reference.

### BACKGROUND

Bronchoscopy is an endoscopic technique involving inserting a bronchoscope into a patient's airways for diagnostic and therapeutic purposes. Diagnostic purposes may include, e.g., viewing abnormalities in the airways, obtaining lung tissue specimens, and evaluating bleeding in the lungs. Therapeutic purposes can include, e.g., removing foreign objects lodged in the airways, laser resection of tumors or benign tracheal and bronchial restrictions, and stent insertion.

### BRIEF SUMMARY

A bronchoscope head in accordance with one or more embodiments includes a main barrel having a distal end and a proximal end. The main barrel defines a working channel extending along a longitudinal axis between an opening at the proximal end and an opening at the distal end. An anesthetic/ventilation port structure extends laterally from and is fixedly connected to the main barrel. The anesthetic/ventilation port structure includes a channel therein in communication with the working channel of the main barrel. A locking mechanism is connected to the distal end of the main barrel and is configured to releasably secure and seal one tube of a set of interchangeable tubes to the main barrel such that a user can rotatably adjust the one tube or the main barrel relative to one another about the longitudinal axis. An end cap is releasably connected to and sealed to the proximal end of the main barrel such that the user can rotatably adjust the end cap or the main barrel relative to one another about the longitudinal axis independently of any rotational adjustments of the main barrel or the one tube relative to each other. The end cap includes one or more working port structures, each working port structure having a channel therein in communication with the working channel of the main barrel and configured for introducing one or more instruments into the main barrel and the one tube connected to the main barrel.

A bronchoscope head in accordance with one or more further embodiments includes a main barrel having a distal end and a proximal end. The main barrel defines a working channel extending along a longitudinal axis between an opening at the proximal end and an opening at the distal end. An anesthetic/ventilation port structure extends laterally from and is fixedly connected to the main barrel. The anesthetic/ventilation port structure includes a channel therein in communication with the working channel of the main barrel. A locking mechanism is connected to the distal end of the main barrel and configured to releasably secure and seal one tube of a set of interchangeable tubes to the main barrel such that a user can rotatably adjust the one tube or the main barrel relative to one another about the longitudinal axis. The locking mechanism comprises an axial thrust bearing component connected to the distal end of the main barrel and a locking cam nut connected to and in-between the axial thrust bearing component and the one tube.

A bronchoscope head in accordance with one or more further embodiments includes a main barrel having a distal end and a proximal end. The main barrel defines a working channel extending along a longitudinal axis between an opening at the proximal end and an opening at the distal end. An anesthetic/ventilation port structure extends laterally from and is fixedly connected to the main barrel. The anesthetic/ventilation port structure includes a channel therein in communication with the working channel of the main barrel. A locking mechanism is connected to the distal end of the main barrel and is configured to releasably secure and seal one tube of a set of interchangeable tubes to the main barrel such that a user can rotatably adjust the one tube or the main barrel relative to one another about the longitudinal axis. A jet Venturi injector comprising a jet Venturi sleeve is fitted over an open end of the anesthetic/ventilation port structure. A jet Venturi tube has a first end at a port in the jet Venturi sleeve and an opposite second end protruding from the channel in the anesthetic/ventilation port structure into the working channel of the main barrel. The second end of the jet Venturi tube has a tip with an opening oriented to supply a substance flowing through the jet Venturi tube into the working channel of the main barrel at an angle of 0 to 90 degrees (more preferably 30 to 45 degrees) relative to the longitudinal axis of the main barrel.

A bronchoscope head with tube adapter in accordance with one or more further embodiments includes a main barrel having a distal end and a proximal end. The main barrel defines a working channel extending along a longitudinal axis between an opening at the proximal end and an opening at the distal end. An anesthetic/ventilation port structure extends laterally from and is fixedly connected to the main barrel. The anesthetic/ventilation port structure includes a channel therein in communication with the working channel of the main barrel. A locking mechanism is connected to the distal end of the main barrel and is configured to releasably secure and seal one tube of a first set of interchangeable tubes to the main barrel. Each of the first set of interchangeable tubes includes a same sized male cam connector at a proximal end of the tube. The locking mechanism includes a female cam nut adapted to be coupled to the male cam connector of any of the first set of interchangeable tubes. An adapter coupling has a male cam connector at one end thereof and a female cam nut at an opposite end thereof. The male cam connector of the adapter coupling is the same size as the male cam connector of the first set of interchangeable tubes and is coupled to the female cam nut of the locking mechanism such that a user can rotatably adjust the adapter coupling and the main barrel relative to one another about the longitudinal axis. The female cam nut of the adapter coupling is configured to releasably secure and seal one tube of a second set of interchangeable tubes. Each of the second set of interchangeable tubes includes a same sized male connector at a proximal end of the tube that is smaller than the same sized male cam connector at the proximal end of each of the first set of interchangeable tubes.

A bronchoscope head in accordance with one or more embodiments includes a main barrel having a distal end and a proximal end. The main barrel defines a working channel extending along a longitudinal axis between an opening at the proximal end and an opening at the distal end. The bronchoscope head also includes an anesthetic/breathing air structure having a distal end connected to the proximal end of the main barrel. The anesthetic/breathing air structure includes an anesthetic port for flow of an anesthetic fluid through the anesthetic/breathing air structure into the working channel of the main barrel, and a breathing air port for flow of a breathing air mixture through the anesthetic/breathing air structure into the working channel of the main barrel. A Venturi tube diffuser/atomizer nozzle in the anesthetic/breathing air structure is connected to the anesthetic port for diffusing or atomizing the anesthetic fluid to improve mixing of the anesthetic fluid with the breathing air mixture.

In accordance with one or more further embodiments, a method is disclosed of mixing an anesthetic fluid and a breathing air mixture in a bronchoscope. The method includes the steps of (a) introducing a stream of the breathing air mixture into a working channel of a main barrel of the bronchoscope; (b) introducing the anesthetic fluid into the working channel of the main barrel of the bronchoscope; and (c) atomizing the anesthetic fluid when the anesthetic fluid is a liquid or diffusing the anesthetic fluid when the anesthetic fluid is a gas into the stream of the breathing air mixture to improve mixing of the anesthetic fluid with the breathing air mixture.

### BRIEF DESCRIPTION OF THE DRAWINGS

FIG. 1 illustrates an exemplary bronchoscope with two levels of adjustable rotation in accordance with one or more embodiments.

FIG. 2 is a partial cross section view of the end cap of the FIG. 1 bronchoscope.

FIG. 3 is an enlarged cross-sectional view illustrating the bearings of the locking mechanism of the FIG. 1 bronchoscope.

FIGS. 4A and 4B illustrate a jet Venturi injector in accordance with one or more embodiments for a bronchoscope.

FIG. 4C illustrates a reduction in working space from jet Venturi injectors in the prior art.

FIGS. 5A and 5B illustrate a bronchoscope in accordance with one or more embodiments with a tube adapter designed to enable bronchial and tracheal tubes with smaller diameters to be used on standard large bore bronchoscope systems.

FIGS. 6A and 6B illustrate use of a Venturi tube diffuser and atomizer, respectively, in a bronchoscope in accordance with one or more embodiments.

Like reference numerals in the figures represent like elements.

### DETAILED DESCRIPTION

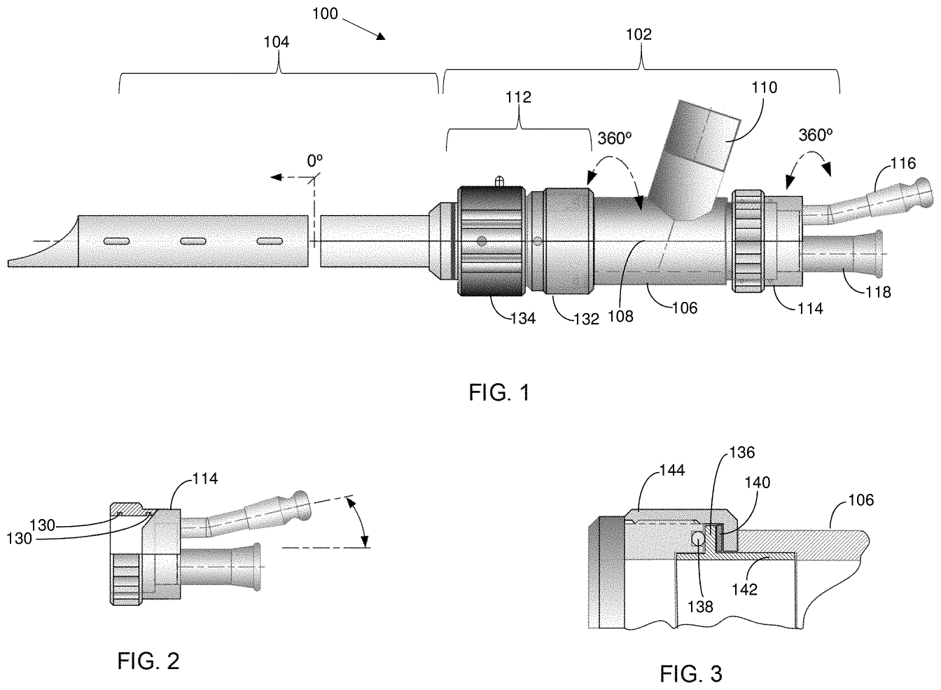

FIG. 1 illustrates an exemplary bronchoscope 100 with two levels of sealed adjustable rotation in accordance with one or more embodiments. The bronchoscope includes a universal bronchoscope head 102, which is attached to a tube 104 that can be is inserted into a patient's airways. The tube 104 is part of a set of interchangeable bronchial and/or tracheal tubes of varying diameters, which can be distinguished, e.g., by color coding.

The bronchoscope head 102 includes a main barrel 106, which defines a working channel therein extending along a longitudinal axis 108 between an opening at the proximal end of the bronchoscope head 102 and an opening at the distal end of the bronchoscope head 102.

An anesthetic/ventilation port structure 110 extends laterally from and is fixedly connected to the main barrel 106. The anesthetic/ventilation port structure 110 includes a channel therein in communication with the working channel of the main barrel 106.

A locking mechanism 112 is connected to the distal end of the main barrel 106 and is configured to releasably secure and seal the tube 104 to the main barrel 106. The locking mechanism 112 is connected to the main barrel 106 such that a user can rotatably adjust the tube 104 or the main barrel 106 relative to one another preferably 360° in either direction about the longitudinal axis 108. The locking mechanism 112 enables the tube 104 to be rotated by a user by twisting the tube 104 or barrel 106 relative to each other. This allows practitioners to better place and freely reposition the anesthetic/ventilation connection so that it will have minimal obstruction during the procedure.

As shown in FIG. 1, the locking mechanism 112 comprises an axial thrust bearing component 132 connected to the distal end of the main barrel 106 and a locking cam nut 134 connected to and being positioned in-between the axial thrust bearing component 132 and the tube 104. FIG. 3 is an enlarged cross-sectional view of a portion of the locking mechanism 112. The axial thrust bearing component 132 includes an axial thrust bearing member 136 having a first seal 138 on one side thereof facing the locking cam nut 134 and a second seal 140 on opposite sides thereof facing the main barrel 106 to provide frictional resistance to inhibit inadvertent movement of the main barrel 106 or the tube 104 relative to one another. In one or more embodiments, the amount of torque needed to move the main barrel 106 or the tube 104 relative to one another is 0.5 to 3 inch-pounds. In one or more particular embodiments, the torque is 1 inch-pound.

In the illustrated embodiment, the first seal 138 comprises an O-ring seal, and the second seal 140 comprises a slip ring seal. In one or more embodiments, the slip ring seal comprises Teflon, Delrin, or similar material.

The axial thrust bearing component 132 comprises a tubular member having one end 142 press-fitted in the main barrel 106, and an opposite end having the axial thrust bearing member 136 extending radially from the tubular member.

The axial thrust bearing component 132 includes a sleeve nut 144 having one end facing the slip ring seal 140, and an opposite end screwed onto a portion of the locking cam nut 134.

The locking mechanism 112 thereby securely connects the main bronchoscope head 102 to the bronchial or tracheal tubes 104. The connection is airtight, inhibiting leakage out of the device.

As shown in FIG. 1, an end cap 114 is releasably connected to and sealed to the proximal end of the main barrel 106 such that the user can rotatably adjust the end cap 114 or the main barrel 106 relative to one another preferably 360° in either direction about the longitudinal axis 108. The rotatability of the end cap 114 is independent of any rotational adjustments of the main barrel 106 or the tube 104 relative to each other through the locking mechanism 112. This allows users to have an additional degree of freedom in rotatably adjusting the elements of the bronchoscope, enabling better placement and repositioning of instruments in the main barrel 106.

The end cap 114 includes one or more working port structures 116, 118. Each working port structure has a channel therein in communication with the working channel of the main barrel 106. The working port structures 116, 118 enable one or more instruments to be introduced into the main barrel 106 and the tube 104 connected to the main barrel 106. Such instruments can include, e.g., optical scopes, light sources, biopsy needles, forceps, suction catheters, laser fibers, and stent delivery systems.

The working port structure 116 is preferably oriented at a service angle between 15-30 degrees relative to the longitudinal axis 108 of the working channel in the main barrel 106. This service angle is significantly lower than that of prior art devices in which the working ports extend laterally from the main barrel 106 at a steep angle. Having such a steep angle makes it significantly difficult for users to insert and manipulate instruments in the in the bronchoscope.

Additionally, the working port structure 116 is preferably configured such that the bronchoscope has only a single point of curvature for instruments introduced through the working port structure into the working channel of the main barrel 106, which makes it significantly easier to insert and manipulate instruments in the bronchoscope. It also allows use of a greater variety of instruments, especially biopsy needles and forceps, which have a long rigid leading portion that will not significantly bend.

FIG. 2 is a partial cross section view of the end cap 114, illustrating two rotational seals 130 providing an airtight coupling of the end cap 114 to the main barrel 106. The number of seals 130 can be varied as desired. The seals 130 also provide frictional resistance to inhibit inadvertent movement of the end cap 114 relative to the main barrel 106. In one or more embodiments, the amount of torque needed to move the main barrel 106 or the end cap 114 relative to one another can be 0.5 to 3 inch-pounds. In one or more particular embodiments, the torque is 1 inch-pound.

The main barrel 106, the anesthetic/ventilation port structure 110, the locking mechanism 112, and the end cap 114 can comprise a variety of suitable FDA approved materials including, e.g., 316 grade stainless steel or similar high grade stainless steel. The seals and gaskets in the bronchoscope are designed to withstand high temperatures (which can be over 400° F.) and cleaning solvents used in the sterilization processes of medical instruments.

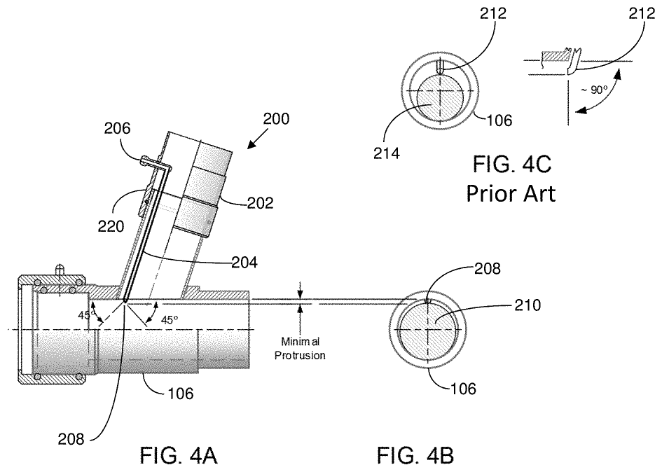

FIGS. 4A and 4B illustrate a jet Venturi injector 200 in accordance with one or more embodiments for a bronchoscope. The jet Venturi injector 200 includes a jet Venturi sleeve 202 fitted over the open end of the anesthetic/ventilation port structure 110 of the main barrel 106. A jet Venturi tube 204 has a first end 206 at a port in the jet Venturi sleeve and an opposite second end 208 protruding from the channel of the anesthetic/ventilation port structure 110 into the working channel of the main barrel 106. The second end of the jet Venturi tube has a tip with an opening oriented to supply a substance (e.g., an anesthetic) flowing through the jet Venturi tube into the working channel of the main barrel 106 at an angle of, e.g., 0 to 90 degrees (more preferably 30 to 45 degrees) relative to the longitudinal axis 108 of the main barrel 106.

The jet Venturi tube protrudes into the working channel of the main barrel 106 only by a small distance, e.g., 0 mm to 2 mm. Accordingly, the jet Venturi tube does not significantly reduce the working area 210 of the main barrel 106. In one or more embodiments, the working area of the working channel is reduced by no more than 10% by the jet Venturi tube.

Prior art jet Venturi tubes are known to protrude deep into the working space of main barrels as shown, e.g., in FIG. 4C, which shows the tip 212 of a prior art jet Venturi tube protruding a greater distance into the working channel of the main barrel 106 and thereby providing a significantly reduced working area 214.

By providing a larger working area in the working channel, users can more easily insert, manipulate, and operate instruments in the channel. By having the nozzle protruding into the working channel by only a small distance significantly reduces the possibility of instruments (especially optical and mechanical instruments) from getting damaged from impact with the nozzle. Additionally, a nozzle that does not deeply protrude into the working channel is less likely to bend or become damaged from handling during the sterilization process or during insertion of instruments into the working channel.

The jet Venturi injector 200 also includes a high-temperature radial seal 220, which inhibits air or a breathing air mixture from escaping or leaking during the procedure. It also allows the tube to be fine adjusted for optimal flow results.

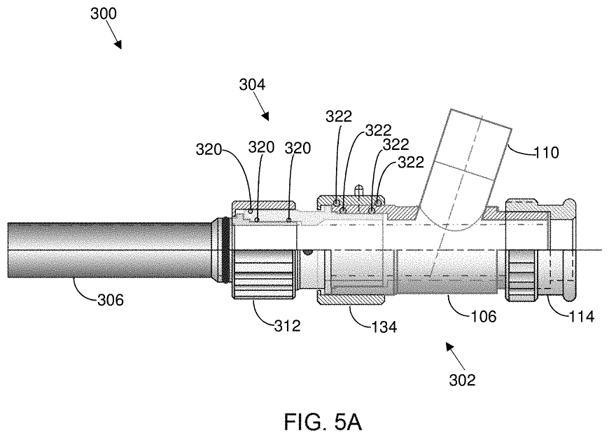

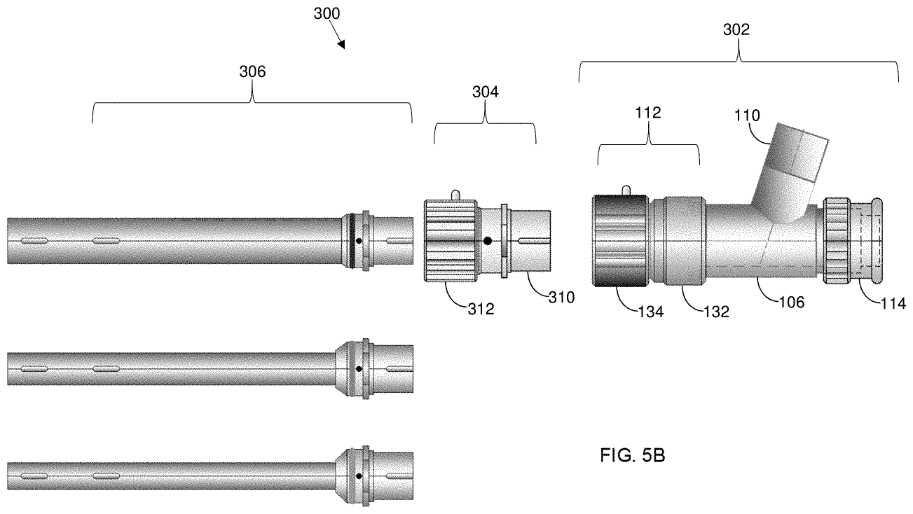

FIGS. 5A and 5B illustrate a bronchoscope 300 in accordance with one or more embodiments with a tube adapter designed to enable bronchial and tracheal tubes 306 with smaller diameters to be used on large bore bronchoscope systems. FIG. 5A is a partial cross sectioned view of an assembled bronchoscope, and FIG. 5B shows the components separated for purposes of illustration.

The bronchoscope 300 includes a bronchoscope head 302 and an adapter coupling 304 connecting a tube 306 to the bronchoscope barrel 106.

The bronchoscope head 302 which can be the same as or similar to the bronchoscope head 102 in FIG. 1. The bronchoscope head 302 includes a main barrel 106, an anesthetic/ventilation port structure 110, and a locking mechanism 112 as previously described. Additionally, similar to the FIG. 1 embodiment, the locking mechanism 112 comprises an axial thrust bearing component 132 connected to the distal end of the main barrel 106 and a locking cam nut 134 connected to and being positioned in-between the axial thrust bearing component 132 and the adapter coupling 304. The locking mechanism 112 securely connects the main bronchoscope head 302 to the adapter coupling 304. The connection is airtight, inhibiting leakage out of the device.

The locking mechanism 112 is designed to be connected directly to a set of large diameter interchangeable tubes (e.g., diameters above 13.2 mm). Each of the large diameter interchangeable tubes includes a same sized male cam connector at a proximal end of the tube. The female cam nut 134 of the locking mechanism 112 is adapted to be coupled to the male cam connector of any of the set of large diameter interchangeable tubes. However, as shown in FIGS. 5A and 5B, the female cam nut 134 is connected to the adapter coupling 304.

The adapter coupling 304 has a male cam connector 310 at one end and a female cam nut 312 at an opposite end. The male cam connector 310 is the same size as the male cam connector of the first set of large diameter interchangeable tubes and is coupled to the female cam nut 312 of the locking mechanism 112 such that a user can rotatably adjust the adapter coupling 304 and the main barrel 106 relative to one another about the longitudinal axis 108. The female cam nut 312 of the adapter coupling 304 is configured to releasably secure and seal one tube 306 of a set of small diameter interchangeable tubes (e.g., diameters ranging from 6 mm to 13.2 mm). Each of the set of small diameter interchangeable tubes 306 includes a same sized male connector at a proximal end of the tube 306 that is smaller than the same sized male cam connector at the proximal end of each of the set of large diameter interchangeable tubes.

The adapter coupling 304 sealed to the tube 306 by seals 320. The adapter coupling 304 sealed to the locking mechanism 112 by seals 322.

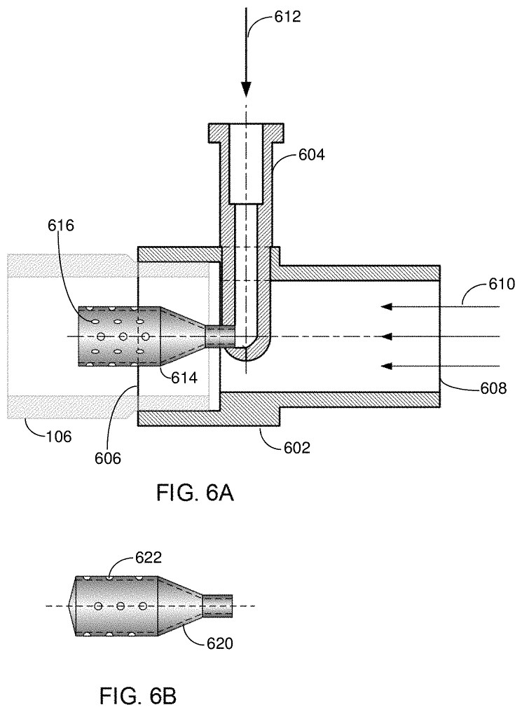

FIGS. 6A and 6B illustrate use of a Venturi tube diffuser and atomizer, respectively, in a bronchoscope to improve mixing of anesthetics with a breathing air mixture in accordance with one or more embodiments.

As shown in FIG. 6A, an anesthetic/breathing air structure 602 includes an anesthetic port 604 having an input connector (e.g., a Luer input connector). The an anesthetic/breathing air structure 602 includes a distal end 606 with a universal barrel connector adapted to be fitted over the open end of the main barrel 106 [is this correct?] of a bronchoscope. The anesthetic/breathing air structure 602 also includes a proximal end 608 through which a stream of breathing air mixture 610 is received for flow into the working channel of the main barrel 106. A flow of anesthetics 612 is received in the port 604 also for flow into the working channel of the main barrel 106.

In accordance with one or more embodiments, a Venturi tube diffuser nozzle 614 is connected to the end of the port 604 and extends into the working channel of the main barrel 106. The diffuser nozzle 614 is open ended and includes a plurality of openings 616 designed to diffuse a gas flow of anesthetics 612 into the breathing air mixture 610. The body of the diffuser nozzle 614 containing the openings 616 is an enlarged and constricts the flow of breathing gases through the anesthetic/breathing air structure 602 and the main barrel 106. This flow constriction increases the gas flow rate and at the same time reduces pressure around the diffuser nozzle 614. The lower pressure causes anesthesia 612 to be drawn out of the nozzle 614 through the openings 616, which diffuses the anesthesia 612 and improves mixing of the anesthesia 612 with the breathing air 610.

FIG. 6B illustrates an atomizer nozzle 620, which can be used in place of the diffuser nozzle 614 when liquid anesthesia is to be introduced into the bronchoscope. The atomizer nozzle 620 includes a plurality of openings 622, through which the liquid anesthesia is drawn out by the flow of breathing air 610. The openings 622 are designed to cause the liquid anesthesia to be atomized for improved mixing with the breathing air 610. Unlike the diffuser nozzle 614, the atomizer nozzle 620 is close ended, i.e., the liquid anesthesia flows entirely through the openings 622.

The bronchoscopes described herein in accordance with various embodiments comprise materials designed to withstand high temperature sterilization procedures and exposure to cleaning solvents. The surfaces of the components are noncorrosive and smooth (without sharp edges and preferably with an ultrafine uniform texture).

Having thus described several illustrative embodiments, it is to be appreciated that various alterations, modifications, and improvements will readily occur to those skilled in the art. Such alterations, modifications, and improvements are intended to form a part of this disclosure, and are intended to be within the spirit and scope of this disclosure. While some examples presented herein involve specific combinations of functions or structural elements, it should be understood that those functions and elements may be combined in other ways according to the present disclosure to accomplish the same or different objectives. In particular, acts, elements, and features discussed in connection with one embodiment are not intended to be excluded from similar or other roles in other embodiments. Additionally, elements and components described herein may be further divided into additional components or joined together to form fewer components for performing the same functions.

Accordingly, the foregoing description and attached drawings are by way of example only, and are not intended to be limiting.

1. A bronchoscope head, comprising:
a main barrel having a distal end and a proximal end, the main barrel defining a working channel extending along a longitudinal axis between an opening at the proximal end and an opening at the distal end;
an anesthetic/ventilation port structure extending laterally from and being fixedly connected to the main barrel, the anesthetic/ventilation port structure including a channel therein in communication with the working channel of the main barrel; and
a locking mechanism connected to the distal end of the main barrel and configured to releasably secure and seal one tube of a set of interchangeable tubes to the main barrel such that a user can rotatably adjust the one tube or the main barrel relative to one another about the longitudinal axis, wherein the locking mechanism comprises an axial thrust bearing component connected to the distal end of the main barrel and a locking cam nut connected to and in-between the axial thrust bearing component and the one tube.

2. The bronchoscope head of claim 1, wherein the axial thrust bearing component includes an axial thrust bearing member having a first seal on one side thereof facing the locking cam nut and a second seal on opposite sides thereof facing the main barrel to provide frictional resistance to inhibit inadvertent movement of the main barrel or the one tube relative to one another.

3. The bronchoscope head of claim 2, wherein the first seal comprises an O-ring seal, and the second seal comprises a slip ring seal.

4. The bronchoscope head of claim 3, wherein the slip ring seal comprises Teflon or Delrin material.

5. The bronchoscope head of claim 2, wherein the axial thrust bearing component comprises a tubular member having one end press-fitted in the main barrel, and an opposite end having the axial thrust bearing member extending radially from the tubular member.

6. The bronchoscope head of claim 5, wherein the axial thrust bearing component includes a sleeve nut having one end facing the slip ring seal, and an opposite end screwed onto a portion of the locking cam nut.

7. The bronchoscope head of claim 1, wherein the set of interchangeable tubes comprises bronchial tubes and/or tracheal tubes.

8. The bronchoscope head of claim 1, further comprising an end cap releasably connected to and sealed to the proximal end of the main barrel such that the user can rotatably adjust the end cap or the main barrel relative to one another about the longitudinal axis independently of any rotational adjustments of the main barrel or the one tube relative to each other, the end cap including one or more working port structures, each working port structure having a channel therein in communication with the working channel of the main barrel and configured for introducing one or more instruments into the main barrel and the one tube connected to the main barrel.

9. The bronchoscope head of claim 1, further comprising a jet Venturi injector comprising a jet Venturi sleeve fitted over an open end of the anesthetic/ventilation port structure and a jet Venturi tube having a first end at a port in the jet Venturi sleeve and an opposite second end protruding from the channel in the anesthetic/ventilation port structure into the working channel of the main barrel, said second end of the jet Venturi tube having a tip with an opening oriented to supply a substance flowing through the jet Venturi tube into the working channel of the main barrel at an angle of 0 to 90 degrees relative to the longitudinal axis of the main barrel.

10. The bronchoscope head of claim 9, wherein the jet Venturi tube protrudes into the working channel of the main barrel only by a distance of 0 mm to 2 mm.

11. The bronchoscope head of claim 1, further comprising an adapter coupling for enabling different sized tubes to be secured to the main barrel, said adapter coupling having a male cam connector at one end thereof and a female cam nut at an opposite end thereof, wherein the male cam connector of the adapter coupling is coupled to a female cam nut of the locking mechanism such that a user can rotatably adjust the adapter coupling and the main barrel relative to one another about the longitudinal axis, and wherein the female cam nut of the adapter coupling is configured to releasably secure and seal said one tube.
